(19) United States
(12) Patent Application Publication
(10) Pub. No.: US 2010/0256199 A1
(43) Pub. Date: Oct. 7, 2010

(54) Crystalline form of an Antimalarial Compound

(75) Inventors: Pingyun Y. Chem, Research Triangle Park, NC (US); Ricky Couch, Research Triangle Park, NC (US)

Correspondence Address: GlaxoSmithKline;GLOBAL PATENTS -US, UW2220, P. O. BOX 1539, KING OF PRUSSIA, PA, 19406-0939, US

(73) Assignee: Glaxo Group Limited

(21) Appl. No.: 12/744,333

(22) PCT Filed: Nov. 27, 2008
(86) PCT No.: PCT/EP08/66360

§ 371 Date: May 24, 2010

Related U.S. Application Data
(60) Provisional application No. 60/991,282, filed on Nov. 30, 2007.

(51) Int. Cl. A61K 31/4418; C07D 213/68; A61P 33/06

(52) U.S. Cl. 514/345; 546/302

(57) ABSTRACT

The present invention relates to a polymorphic form of the compound 3-chloro-6-(hydroxymethyl)-2-methyl-5-[4-({4-[(trifluoromethyl)oxy]phenyl}oxy)phenyl]-4(1H)-pyridinone, methods of preparing it, pharmaceutical compositions and medicaments containing the same, and use of such polymorph, compositions and medicaments in the treatment or prevention of a condition caused by certain parasitic infections such as malaria, and in particular a condition caused by infection by Plasmodium falciparum

### FIELD OF THE INVENTION

The present invention relates to a polymorphic form of the compound 3-chloro-6-(hydroxymethyl)-2-methyl-5-[4-({4-[(trifluoromethyl)oxy]phenyl}oxy)phenyl]-4(1H)-pyridinone, methods of preparing it, pharmaceutical compositions and medicaments containing the same, and use of such polymorph, compositions and medicaments in the treatment or prevention of a condition caused by certain parasitic infections such as malaria, and in particular a condition caused by infection by Plasmodium falciparum.

### BACKGROUND TO THE INVENTION

Parasitic protozoal infections are responsible for a wide variety of diseases of medical and veterinary importance, including malaria in man and various coccidioses in birds, fish and mammals. Many of the diseases are life-threatening to the host and cause considerable economic loss in animal husbandry, such as species of Eimeria, Theileria, Babesia, Cryptosporidium, Toxoplasma (such as Toxoplasma brucei, African sleeping sickness and Toxoplasma cruzi, Chagas disease) and Plasmodium (such as Plasmodium falciparum), and the Mastigophora such as species of Leishmania (such as Leishmania donovani). Another parasitic organism of increasing concern is Pneumocytis carinii, which can cause an often fatal pneumonia in immunodeficient or immunocompromised hosts, including those infected with HIV.

Malaria is one of the major disease problems of the developing world. The most virulent malaria-causing parasite in humans is the parasite Plasmodium falciparum, which is the cause of hundreds of millions of cases of malaria per annum, and is thought to cause over 1 million deaths each year, Breman, J. G., et al., (2001) Am. Trop. Med. Hyg. 64, 1-11. One problem encountered in the treatment of malaria is the build-up of resistance by the parasite to available drugs. Thus, there is a need to develop new antimalarial drugs.

A group of 3,5-dihalo-2,6-dialkyl-4-pyridinol derivatives (the tautomeric form of 4-pyridones) is described in U.S. Pat. No. 3,206,358 as having anticoccidial activity.

European Patent Application EP123239 discloses combinations of the aforementioned 4-pyridinol derivatives with antiprotozoal naphthoquinones, e.g. antimalarial naphthoquinones, in a potentiating ratio.

PCT Patent Application WO 91/13873 A1 discloses a class of 4-pyridone derivatives which exhibit activity against protozoa, in particular against the malarial parasite Plasmodium falciparum, and species of Eimeria as well as the parasitic organism Pneumocytis carinii.

PCT Patent Application WO 2006/094799 A2 discloses certain 4-pyridone (4-pyridinone) derivatives and their use in chemotherapy of certain parasitic infections such as malaria, and in particular infection by Plasmodium falciparum.

PCT Patent Application No. PCT/EP2007/055188, published as WO 2007/138048, discloses certain 4-pyridone (4-pyridinone) derivatives and their use in chemotherapy of certain parasitic infections such as malaria, and in particular infection by Plasmodium falciparum.

A particularly preferred 4-pyridone derivative for use in chemotherapy of certain parasitic infections such as malaria, and in particular infection by Plasmodium falciparum, is 3-chloro-6-(hydroxymethyl)-2-methyl-5-[4-({4-[(trifluoromethyl)oxy]phenyl}oxy)phenyl]-4(1H)-pyridin-one according to Formula (I), and pharmaceutically acceptable salts, thereof.

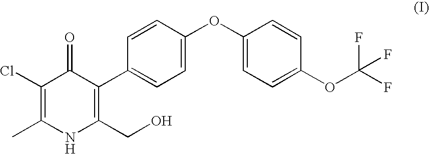

PCT Patent Application No. PCT/EP2007/055188, published as WO 2007/138048, (the contents of which are incorporated by reference) describes the synthesis of the compound of Formula (I) as a non-solvated, free base form. The compound of Formula (I) thus obtained is designated “Form 1” and is a crystalline, white powder.

The compound of Formula (I) as Form 1 is characterised by an XRD pattern expressed in terms of 2 theta angles and obtained with a diffractometer using copper Kα-radiation (45 kV/40 mA) at 0.02 °2θ step, according to the procedures described herein, wherein the XRD pattern comprises 2 theta angles (°2θ), with a margin of error of approximately ±0.1 degrees, at 5.6, 11.2, 14.1, 14.3, 16.3, 16.8, 18.5, 20.7, 21.0, 21.2, 22.2, 22.5, 23.4, 24.9, 28.3, 28.5, 31.2, 31.5, 32.9, 34.2, 37.1 and 40.0 degrees, which correspond respectively to d-spacings at 15.7, 7.9, 6.3, 6.2, 5.4, 5.3, 4.8, 4.3, 4.2, 4.2, 4.0, 3.9, 3.8, 3.6, 3.2, 3.1, 2.9, 2.8, 2.7, 2.6, 2.4 and 2.2 Angstroms (Å).

The compound of Formula (I) as Form 1 is further characterised in that it provides substantially the same X-ray powder diffraction (XRD) pattern as FIG. 5, wherein the XRD pattern is expressed in terms of 2 theta angles and obtained with a diffractometer using copper Kα-radiation (45 kV/40 mA) at 0.02 °2θ step according to the procedures described herein.

The compound of Formula (I) as Form 1 is also characterised by a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks, with a margin of error of approximately ±1 cm⁻¹, at 349, 376, 407, 595, 604, 634, 811, 868, 1049, 1157, 1167, 1208, 1296, 1342, 1452, 1507, 1525, 1580, 1603, 1616, 2924, 3071 and 3084 cm⁻¹.

The compound of Formula (I) as Form 1 is further characterised in that it provides substantially the same Raman spectrum as FIG. 6, wherein the Raman spectrum is obtained using a Fourier Transform (FT) Raman spectrometer equipped with a 1064 nm excitation laser and a liquid nitrogen cooled Ge detector at spectral resolution of 4 cm⁻¹ according to the procedures described herein.

The compound of Formula (I) as Form 1 is further characterised in that it provides substantially the same thermogravimetric analysis (TGA) curve as FIG. 8, wherein the TGA was performed using open platinum pan at a heating rate of 15° C. per minute according to the procedures described herein.

Polymorphism is defined as the ability of an element or compound to crystallise in more than one distinct crystalline phase. Thus polymorphs are distinct solids sharing the same molecular formula, however since the properties of any solid depends on its structure, different polymorphs may exhibit distinct physical properties such as different solubility profiles, different melting points, different dissolution profiles, different thermal and/or photostability, different shelf life, different suspension properties and different physiological absorption rate. Inclusion of a solvent in the crystalline solid leads to solvates, and in the case of water as a solvent, hydrates.

Polymorphic forms of a compound may be distinguished from one another and from an amorphous phase of the compound by methods including but not limited to x-ray diffraction (XRD), infra-red spectroscopy (IR), Raman spectroscopy, differential scanning calorimetry (DSC) and solid state nuclear magnetic resonance spectroscopy (SSNMR).

### SUMMARY OF THE INVENTION

### Form 2

The present invention provides a polymorph of the compound of Formula (I) designated “Form 2”.

As a first aspect, the present invention provides crystalline compound of Formula (I) (Form 2) characterised by substantially the same Raman spectrum as FIG. 1, wherein the Raman spectrum is obtained using a Fourier Transform (FT) Raman spectrometer equipped with a 1064 nm excitation laser and a liquid nitrogen cooled Ge detector at spectral resolution of 4 cm¹ according to the procedures described herein.

As a second aspect, the present invention provides crystalline compound of Formula (I) (Form 2) characterised by a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks at five or more positions selected from the group consisting of: 364, 414, 429, 587, 600, 642, 811, 1074, 1153, 1167, 1209, 1270, 1346, 1527, 1602, 1617, 2937, 3057, 3071 and 3087 cm⁻¹.

As a third aspect, the present invention provides crystalline compound of Formula (I) (Form 2) characterised by a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks at 364, 414, 429, 587, 600, 642, 811, 1074, 1153, 1167, 1209, 1270, 1346, 1527, 1602, 1617, 2937, 3057, 3071 and 3087 cm⁻¹.

As a fourth aspect, the present invention provides crystalline compound of Formula (I) (Form 2) characterised by a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks at 364, 414, 429, 587, 1074, 1270, 1527, 2937 and 3087 cm⁻¹.

As a fifth aspect, the present invention provides crystalline compound of Formula (I) (Form 2) characterised by a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks at 414, 429, 587, 1270 and 2937 cm⁻¹.

As a sixth aspect, the present invention provides crystalline compound of Formula (I) (Form 2) characterised by substantially the same X-ray powder diffraction (XRD) pattern as FIG. 2, wherein the XRD pattern is expressed in terms of 2 theta angles and obtained with a diffractometer using copper Kα-radiation (45 kV/40 mA) at 0.02 °2θ step according to the procedures described herein.

As a seventh aspect, the present invention provides crystalline compound of Formula (I) (Form 2) characterised by an XRD pattern expressed in terms of 2 theta angles and obtained with a diffractometer using copper Kα-radiation (45 kV/40 mA) at 0.02 °2θ step, according to the procedures described herein, wherein the XRD pattern comprises 2 theta angles at four or more positions selected from the group consisting of: 5.0, 10.1, 14.2, 15.1, 16.4, 17.7, 18.9, 19.6, 19.8, 20.0, 20.3, 20.9, 22.5, 23.3, 23.6, 23.7, 25.4, 26.0, 26.5, 28.0, 33.9, 37.5, 39.1 and 40.3 degrees, which correspond respectively to d-spacings at 17.6, 8.8, 6.2, 5.8, 5.4, 5.0, 4.7, 4.5, 4.5, 4.4, 4.4, 4.2, 3.9, 3.8, 3.8, 3.7, 3.5, 3.4, 3.4, 3.2, 2.6, 2.4, 2.3 and 2.2 Angstroms (Å).

As an eighth aspect, the present invention provides crystalline compound of Formula (I) (Form 2) characterised by an XRD pattern expressed in terms of 2 theta angles and obtained with a diffractometer using copper Kα-radiation (45 kV/40 mA) at 0.02 °2θ step, according to the procedures described herein, wherein the XRD pattern comprises 2 theta angles at 5.0, 10.1, 14.2, 15.1, 16.4, 17.7, 18.9, 19.6, 19.8, 20.0, 20.3, 20.9, 22.5, 23.3, 23.6, 23.7, 25.4, 26.0, 26.5, 28.0, 33.9, 37.5, 39.1 and 40.3 degrees, which correspond respectively to d-spacings at 17.6, 8.8, 6.2, 5.8, 5.4, 5.0, 4.7, 4.5, 4.5, 4.4, 4.4, 4.2, 3.9, 3.8, 3.8, 3.7, 3.5, 3.4, 3.4, 3.2, 2.6, 2.4, 2.3 and 2.2 Angstroms (Å).

As a ninth aspect, the present invention provides crystalline compound of Formula (I) (Form 2) characterised by an XRD pattern expressed in terms of 2 theta angles and obtained with a diffractometer using copper Kα-radiation (45 kV/40 mA) at 0.02 °2θ step, according to the procedures described herein, wherein the XRD pattern comprises 2 theta angles at 5.0, 10.1, 14.2, 15.1, 16.4, 18.9, 19.6, 20.0, 25.4, 26.0, 26.5 and 28.0 degrees which correspond respectively to d-spacings at 17.6, 8.8, 6.2, 5.8, 5.4, 4.7, 4.5, 4.4, 3.5, 3.4, 3.4 and 3.2 Angstroms (Å).

As a tenth aspect, present invention provides crystalline compound of Formula (I) (Form 2), characterised by an XRD pattern expressed in terms of 2 theta angles and obtained with a diffractometer using copper Kα-radiation (45 kV/40 mA) at 0.02 °2θ step, according to the procedures described herein, wherein the XRD pattern comprises 2 theta angles (°2θ) at 5.0, 10.1, 14.2, 15.1, 16.4, 18.9, 19.6, 20.0, 25.4, 26.0, 26.5 and 28.0 degrees, which correspond respectively to d-spacings at 17.6, 8.8, 6.2, 5.8, 5.4, 4.7, 4.5, 4.4, 3.5, 3.4, 3.4 and 3.2 Angstroms (Å) and further characterised by a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks, with a margin of error of approximately ±1 cm⁻¹, at 364, 414, 429, 587, 1074, 1270, 1527, 2937 and 3087 cm⁻¹.

As an eleventh aspect, the present invention provides crystalline compound of Formula (I) (Form 2) having substantially the same differential scanning calorimetry (DSC) thermogram as FIG. 3 wherein the DSC was performed at a scan rate of 15° C. per minute, using a crimped aluminium pan, according to the procedures described herein.

As a twelfth aspect, the present invention provides crystalline compound of Formula (I) (Form 2) characterised by substantially the same thermogravimetric analysis (TGA) curve as FIG. 4 wherein the TGA was performed using open platinum pan at a heating rate of 15° C. per minute according to the procedures described herein.

As a further aspect, the present invention provides a pharmaceutical composition comprising crystalline compound of Formula (I) (Form 2) according to the present invention. The pharmaceutical composition may further comprise one or more pharmaceutically acceptable excipients.

The crystalline compound of Formula (I) (Form 2) can be useful in the treatment or prevention of a condition caused by certain parasitic infections, such as parasitic protozoal infections by the malarial parasite Plasmodium falciparum, species of Eimeria, Pneumocytis carnii, Trypanosoma cruzi, Trypanosoma brucei or Leishmania donovani.

In a further aspect, the present invention provides a crystalline compound of Formula (I) (Form 2) according to the present invention for use in therapy, particularly in the treatment or prevention of a condition caused by certain parasitic infections such as malaria, and in particular a condition caused by infection by Plasmodium falciparum.

In a further aspect, the present invention provides a crystalline compound of Formula (I) (Form 2) according to the present invention for use in the treatment or prevention of a condition caused by certain parasitic infections such as malaria, and in particular a condition caused by infection by Plasmodium falciparum.

In a further aspect, the present invention discloses a method for the treatment of a human or animal subject suffering from a condition caused by certain parasitic infections such as malaria, and in particular a condition caused by infection by Plasmodium falciparum, comprising administering to said human or animal subject an effective amount of a crystalline compound of Formula (I) (Form 2) according to the present invention. In one aspect, the subject is a human.

In a further aspect, the present invention provides the use of crystalline compound of Formula (I) (Form 2) according to the present invention in the preparation of a medicament for the treatment or prevention of a condition caused by certain parasitic infections such as malaria, and in particular a condition caused by infection by Plasmodium falciparum.

### BRIEF DESCRIPTION OF THE DRAWINGS

FIG. 1. The Raman spectrum of Form 2 of compound of Formula (I) according to the present invention. The x-axis is wavenumbers in cm⁻¹ and the y-axis is intensity in arbitrary units. The Raman spectrum is obtained using an FT Raman spectrometer with a 1064 nm excitation laser and a liquid nitrogen cooled Ge detector at spectral resolution of 4 cm⁻¹ according to the procedures described herein.

FIG. 2. The XRD pattern of Form 2 of compound of Formula (I) according to the present invention. The XRD pattern is expressed in terms of 2 theta angles and obtained with a diffractometer using copper Kα-radiation (45 kV/40 mA) at 0.02 °2θ step according to the procedures described herein.

FIG. 3. The differential scanning calorimetry (DSC) thermogram for Form 2 of compound of Formula (I) according to the present invention. DSC was performed at a scan rate of 15° C. per minute, using a crimped aluminium pan, according to the procedures described herein.

FIG. 4. The thermogravimetric analysis (TGA) curve for Form 2 of compound of Formula (I) according to the present invention. TGA was measured at a scan rate of 15° C. per minute, using open platinum pan according to the procedures described herein.

FIG. 5. The XRD pattern of Form 1 of compound of Formula (I). The XRD pattern is expressed in terms of 2 theta angles and obtained with a diffractometer using copper Kα-radiation (45 kV/40 mA) at 0.02 °2θ step according to the procedures described herein.

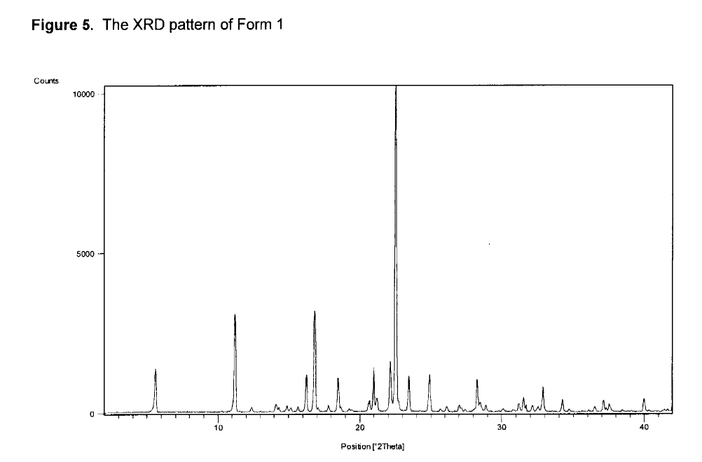

FIG. 6. The Raman spectrum of Form 1 of compound of Formula (I). The x-axis is wavenumbers in cm⁻¹ and the y-axis is intensity in arbitrary units. The Raman spectrum is obtained using an FT Raman spectrometer with a 1064 nm excitation laser and a liquid nitrogen cooled Ge detector at spectral resolution of 4 cm⁻¹ according to the procedures described herein.

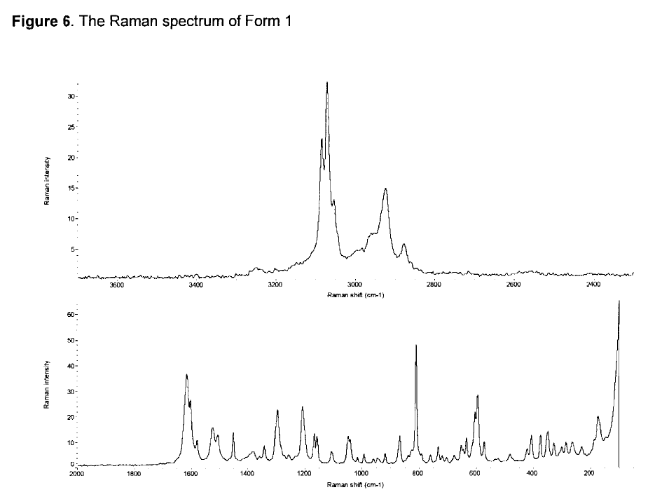

FIG. 7. The differential scanning calorimetry (DSC) thermogram for Form 1 of compound of Formula (I). DSC was performed at a scan rate of 15° C. per minute, using a crimped aluminium pan, according to the procedures described herein.

FIG. 8. The thermogravimetric analysis (TGA) curve for Form 1 of compound of Formula (I). TGA was measured at a scan rate of 15° C. per minute, using open platinum pan according to the procedures described herein.

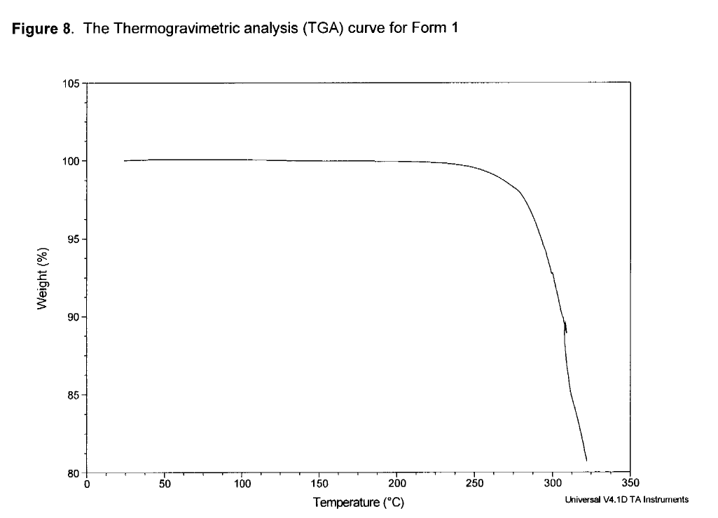

FIG. 9. The XRD pattern of Form 3 of compound of Formula (I). The XRD pattern is expressed in terms of 2 theta angles and obtained with a diffractometer using copper Kα-radiation (45 kV/40 mA) at 0.02 °2θ step according to the procedures described herein, at an elevated temperature of approximately 190° C., with the use of a hot stage.

### DETAILED DESCRIPTION OF THE INVENTION

The present invention provides a crystalline form of compound of Formula (I) (Form 2) exhibiting one or more advantageous pharmaceutical properties or other advantages over other polymorphic forms or over an amorphous phase. The crystalline form of the present invention is thermodynamically more stable than Form 1 at ambient temperatures, for example at 23° C.

### Relative Thermodynamic Stability of Form 1 and Form 2

Forms 1 and 2 of the compound of Formula (I) are both stable at ambient temperature. Competitive ripening experiments between Form 1 and 2 of the compound of Formula (I) were conducted in i) a mixture of acetone and 1-propanol at 23° C.; ii) in acetone at 10° C. and iii) in acetone at 50° C. In each of these ripening experiments, Form 1 converted to Form 2. These results show that Form 2 is thermodynamically more stable than Form 1 between 10° C. and 50° C.

The skilled artisan will appreciate that there are many advantages associated with a more thermodynamically stable polymorph as compared with other polymorphic forms or over an amorphous phase of a given compound. For example, use of a more thermodynamically stable polymorph is expected to minimize the risk of polymorphic form change during the manufacturing process of the compound and during formulation, as well as maximizing the stability and shelf life of the compound of the final and pharmaceutical product.

Further desirable properties of the crystalline form of the present invention (Form 2) include the non-hygroscopic nature of this form.

Polymorphic forms of compound of Formula (I) may be characterised and differentiated using a number of conventional analytical techniques, including but not limited to x-ray powder diffraction (XRD), infra-red spectroscopy (IR), Raman spectroscopy, differential scanning calorimetry (DSC) and solid state nuclear magnetic resonance spectroscopy (SSNMR).

“Form 2 of compound of Formula (I)” as used herein refers to any of:

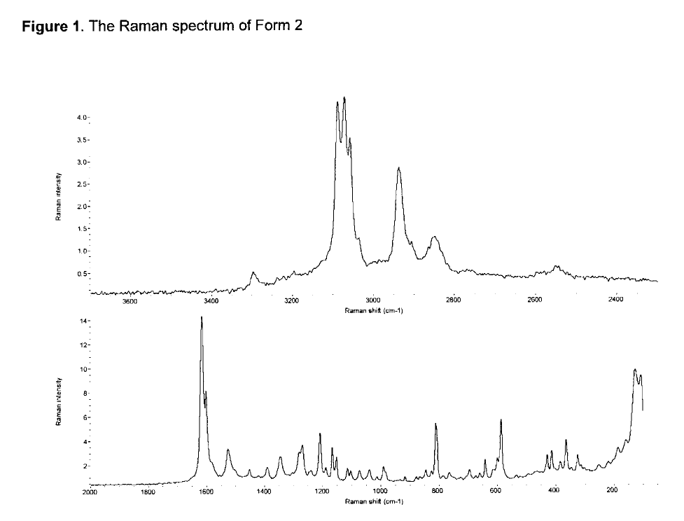

1) a crystalline form of compound of Formula (I) characterised by substantially the same Raman spectrum as FIG. 1, wherein the Raman absorption spectrum is obtained using an FT Raman spectrometer equipped with a 1064 nm excitation laser and a liquid nitrogen cooled Ge detector at spectral resolution of 4 cm⁻¹ according to the procedures described herein.

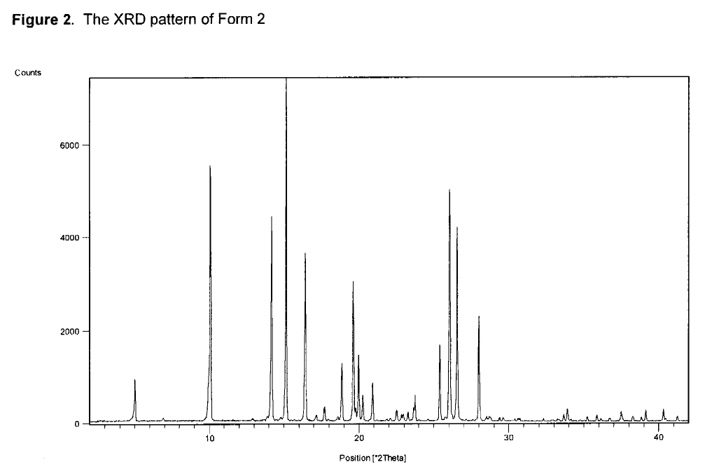

2) a crystalline compound of Formula (I) characterised by substantially the same X-ray powder diffraction (XRD) pattern as FIG. 2, wherein the XRD pattern is expressed in terms of 2 theta angles and obtained with a diffractometer using copper Kα-radiation (45 kV/40 mA) at 0.02 °2θ step according to the procedures described herein.

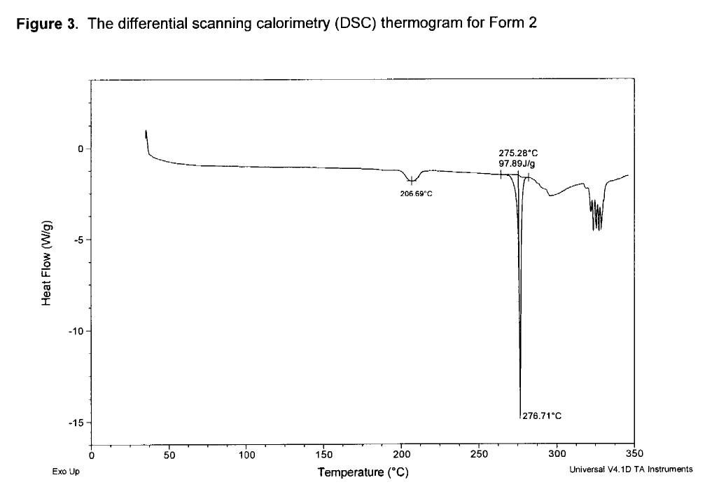

3) a crystalline compound of Formula (I) having substantially the same differential scanning calorimetry (DSC) thermogram as FIG. 3 wherein the DSC was performed at a scan rate of 15° C. per minute, using a crimped aluminium pan, according to the procedures described herein.

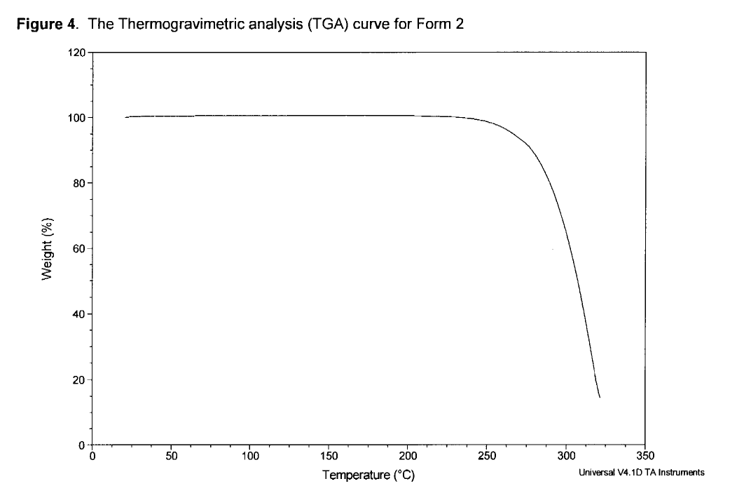

4) a crystalline compound of Formula (I) characterised by substantially the same thermogravimetric analysis (TGA) curve as FIG. 4 wherein the TGA was performed at a scan rate of 15° C., according to the procedures described herein.

### The Raman Spectrum of Form 2

The Raman spectrum of the crystalline form of compound of Formula (I) according to the present invention (i.e., Form 2) can be determined using conventional equipment and techniques known to those skilled in the art of analytical chemistry and physical characterisation. The Raman spectrum of FIG. 1 was obtained on an FT Raman spectrometer equipped with a 1064 nm excitation laser and a liquid nitrogen cooled Ge detector at spectral resolution of 4 cm⁻¹. The wavenumber in cm⁻¹ (x axis) is plotted against the intensity of the scattered light (y axis). Representative peaks observed in the Raman spectrum of Form 2 of compound of Formula (I) are as follows: 364, 414, 429, 587, 600, 642, 811, 1074, 1153, 1167, 1209, 1270, 1346, 1527, 1602, 1617, 2937, 3057, 3071 and 3087 cm⁻¹.

As will be apparent to those skilled in the art, not all of these Raman peaks are necessary to conclusively identify an analyzed sample as Form 2 compound of Formula (I). Form 2 of compound of Formula (I) can be identified by the presence of peaks at 5 or more positions selected form the group consisting of 364, 414, 429, 587, 600, 642, 811, 1074, 1153, 1167, 1209, 1270, 1346, 1527, 1602, 1617, 2937, 3057, 3071 and 3087 cm⁻¹. More particularly, at least peaks at 414, 429, 587, 1270 and 2937 cm⁻¹ are present, in one aspect, 2, 3 or 4 further peaks are present and in a further aspect, all of the foregoing peaks are present.

Slight variations in observed Raman peaks are expected based on the specific spectrometer employed and the analyst's sample preparation technique. Some margin of error is present in each of the peak assignments reported above. The margin of error in the foregoing peak assignments is approximately ±1 cm⁻¹. In one aspect, the margin of error in the foregoing peak assignments is ±1 cm⁻¹.

Since some margin of error is possible in the peak assignments, a useful method of comparing Raman spectra in order to identify the particular form of a sample of compound of Formula (I) is to overlay the Raman spectrum of the sample over the Raman spectrum of another form of a compound of Formula (I), for example that of Form 2. For example, one skilled in the art can overlay a Raman spectrum of a sample of compound of Formula (I), e.g. Form 2, for example as obtained using the methods described herein, over FIG. 1 and, using expertise and knowledge in the art, readily determine whether the Raman spectrum of the sample is substantially the same as the Raman spectrum of Form 2 of compound of Formula (I). If the Raman spectrum is substantially the same as FIG. 1, the sample can be readily and accurately identified as Form 2 of compound or Formula (I).

### The XRD Pattern of Form 2

The X-ray powder diffraction pattern of Form 2 compound of Formula (I) can be determined using conventional techniques and equipment known to those skilled in the art of analytical chemistry and physical characterisation. The diffraction pattern of FIG. 2 was obtained using copper Kα radiation on a Philips X'Pert Pro diffractometer equipped with a Philips X'Celerator Real Time Multi Strip (RTMS) detector. The sample was packed into a zero background holder and scanned from 2 to 40 °2θ using the following acquisition parameters: 40 mA, 45 kV, 0.02 °2θ step, 40 s step time. The sample was spun at 25 rpm during analysis.

A powder sample of Form 2 compound of Formula (I) was used to produce the XRD pattern of FIG. 2. 2 Theta angles in degrees (x-axis) are plotted against peak intensity in terms of the count rate per seconds (y-axis). The XRD pattern for each crystalline form is unique, exhibiting a unique set of diffraction peaks which can be expressed in 2 theta angles (°2θ), d-spacings (Å) and/or relative peak intensities.

2 Theta diffraction angles and corresponding d-spacing values account for positions of various peaks in the XRD pattern. D-spacing values are calculated with observed 2 theta angles and copper Kα wavelength using the Bragg equation. Slight variations in observed 2 theta angles and d-spacings are expected based on the specific diffractometer employed and the analyst's sample preparation technique. More variation is expected for the relative peak intensities. Large variations of relative peak intensities may be observed due to preferred orientation resulting from differences in crystal morphology. Variations in observed 2 theta angles and d-spacings may also be observed depending on the temperature at which the values are measured. Identification of the exact crystal form of a compound should be based primarily on observed 2 theta angles or d-spacings with lesser importance place on relative peak intensities. To identify Form 2 compound of Formula (I) certain characteristic 2 theta angle peaks occur at 5.0, 10.1, 14.2, 15.1, 16.4, 18.9, 19.6, 20.0, 25.4, 26.0, 26.5 and 28.0 degrees, which correspond respectively to d-spacings at 17.6, 8.8, 6.2, 5.8, 5.4, 4.7, 4.5, 4.4, 3.5, 3.4, 3.4 and 3.2 Angstroms (Å).

Although one skilled in the art can identify Form 2 from these characteristic 2 theta angle peaks or d-spacings, in some circumstances it may be desirable to rely upon additional 2 theta angles or d-spacings for the identification of Form 2 compound of Formula (I).

Form 2 compound of Formula (I) typically exhibits 2 theta angle peaks in addition to the foregoing peaks. For example, Form 2 compound of Formula (I) may be further characterised by additional 2 theta angle peaks at essentially the following positions: 17.7, 19.8, 20.3, 20.9, 22.5, 23.3, 23.6, 23.7, 33.9, 37.5, 39.1 and 40.3 degrees which correspond respectively to d-spacings at 5.0, 4.5, 4.4, 4.2, 3.9, 3.8, 3.8, 3.7, 2.6, 2.4, 2.3 and 2.2 Angstroms (Å).

In one aspect at least 4, and more particularly all of the above are employed to identify Form 2 compound of Formula (I).

Based upon the foregoing characteristic features of the XRD pattern of Form 2 compound of Formula (I), one skilled in the art can readily identify Form 2. It will be appreciated by those skilled in the art that the XRD pattern of a sample of Form 2 compound of Formula (I), obtained using the methods described herein, may exhibit additional peaks.

Some margin of error is present in each of the 2 theta angle assignments and d-spacings reported above. The error in determining 2 theta angles and d-spacings decreases with increasing diffraction scan angle or decreasing d-spacing. The margin of error will be dependent on a number of factors, including the exact temperature at which the values are measured. The margin of error in the foregoing 2 theta angles is approximately ±0.1 degrees for each of the foregoing peak assignments. In one aspect, the margin of error in the foregoing 2 theta angles is ±0.1 degrees.

Since some margin of error is possible in the assignment of 2 theta angles and d-spacings, a useful method of comparing XRD patterns in order to identify the particular form of a sample of compound of Formula (I) is to overlay the XRD pattern of the sample over the XRD pattern of a known form of a compound of Formula (I), for example that of Form 2. For example, one skilled in the art can overlay an XRD pattern of a sample of compound of Formula (I), e.g. Form 2, for example as obtained using the method described herein, over FIG. 2 and, using expertise and knowledge in the art, readily determine whether the XRD pattern of the sample is substantially the same as the XRD pattern of Form 2 of compound of Formula (I). If the XRD pattern is substantially the same as FIG. 2, the sample can be readily and accurately identified as Form 2.

### DSC Thermogram for Form 2

Differential Scanning Calorimetry (DSC) was performed on a TA instruments Q1000 Differential Scanning Calorimeter equipped with a refrigerated cooling system.

The DSC thermogram plots the heat flow in watts per second against temperature. The DSC thermogram of Form 2 of compound of Formula (I), as shown in FIG. 3, displays a small endotherm with an onset temperature at approximately 201° C. which corresponds to a polymorphic form change in the solid-state from Form 2 to “Form 3”. The enthalpy of this polymorphic change determined by integrating this endotherm is approximately 20 J/g. The polymorphic form observed at this temperature is designated “Form 3” of the compound of Formula (I). At approximately 276° C., a sharp peak is observed in the DSC thermogram which corresponds to a melt of Form 3 of the compound of Formula (I).

Significant variations in the observed endotherms are expected in respect of the DSC thermogram of Form 2 of the compound of Formula (I), based on the specific instrument and pan configuration employed, the analyst's sample preparation technique, and the sample particle size and weight. In respect of Form 2 of the compound of Formula (I), particularly significant variations are observed based on the sample particle size. Some margin of error is normally present in the endotherms characteristics reported above. In respect of Form 2 of the compound of Formula (I), the margin of error is in the order of ±20° C.

### DSC Thermogram for Form 1

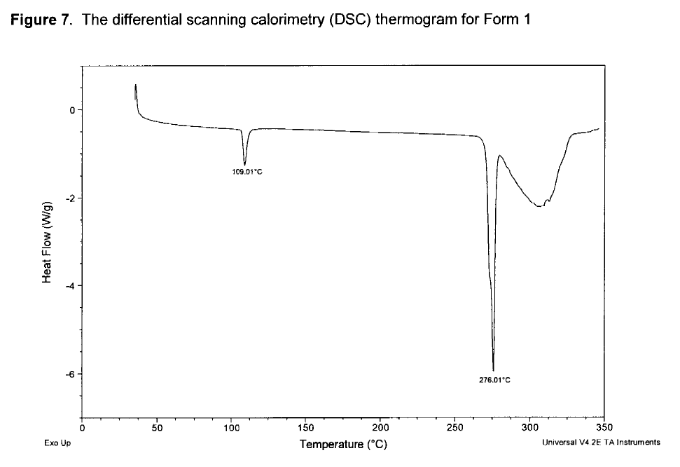

By comparison, the DSC thermogram of Form 1 of compound of Formula (I), as shown in FIG. 7, displays a small endotherm with an onset temperature at approximately 107° C. which corresponds to a polymorphic form change in the solid-state from Form 1 to “Form 3”. The enthalpy of this polymorphic change determined by integrating this endotherm is approximately 10 J/g. The polymorphic form observed at this temperature is designated “Form 3” of the compound of Formula (I), i.e, it is the same polymorphic form as that observed during heating of Form 2, as discussed hereinabove in respect of the DSC thermogram of Form 2. At approximately 276° C., a sharp peak is observed in the DSC thermogram which corresponds to a melt of Form 3 of the compound of Formula (I).

### XRD Pattern of Form 3

The compound of Formula (I) as Form 3, as obtained from Form 1, is characterised by an XRD pattern expressed in terms of 2 theta angles and obtained with a diffractometer using copper Kα-radiation (45 kV/40 mA) at 0.02 °2θ step, according to the procedures described herein, at an elevated temperature of approximately 190° C., wherein the XRD pattern comprises 2 theta angles (°2θ), at 5.9, 11.8, 13.7, 14.2, 14.5, 15.4, 16.3, 17.7, 18.5, 18.9, 19.8, 20.6, 21.4, 22.0, 23.6, 23.9, 28.0, 28.7, 32.8 and 37.5 degrees, which correspond respectively to d-spacings at 15.0, 7.5, 6.5, 6.2, 6.1, 5.7, 5.4, 5.0, 4.8, 4.7, 4.5, 4.3, 4.1, 4.0, 3.8, 3.7, 3.2, 3.1, 2.7 and 2.4 Angstroms (Å).

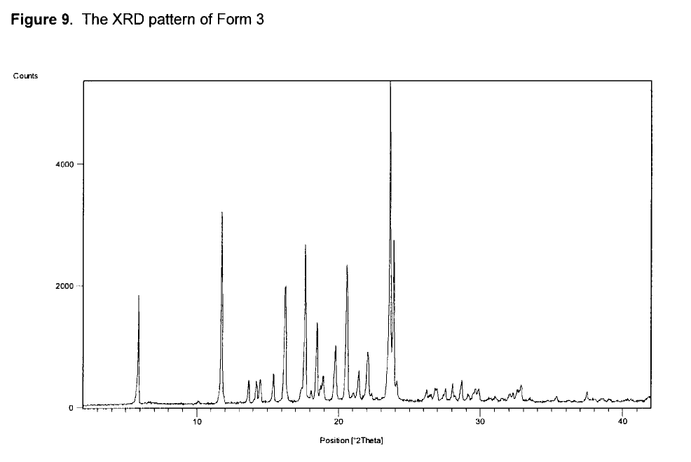

The compound of Formula (I) as Form 3 is further characterised in that it provides substantially the same X-ray powder diffraction (XRD) pattern as FIG. 9, wherein the XRD pattern is expressed in terms of 2 theta angles and obtained with a diffractometer using copper Kα-radiation (45 kV/40 mA) at 0.02 °2θ step according to the procedures described herein, at an elevated temperature of approximately 190° C., with the use of a hot stage.

The skilled artisan will appreciate that the peaks observed in the XRD pattern provided by Form 3 of the compound of Formula (I) may appear different, for example the peaks may be observed at shifted positions, if measured at a temperature which is different from a temperature of approximately 190° C., as employed for the measurement of the XRD pattern reported above.

### Thermogravimetric Analysis (TGA) Curve for Form 2

Thermogravimetric analysis (TGA) was performed using a TA Instruments Thermal Analysis System, Model TGA Q500

The TGA curve (or trace) plots the weight (or weight %) of the sample at different temperatures. The TGA curve (or trace) of Form 2 of compound of Formula (I) displays negligible weight change between ambient and 200° C., which is consistent with Form 2 being a non-solvated form.

Slight variations in the observed curve (trace) is expected based on the specific instrument and pan configuration employed, the analyst's sample preparation technique, the sample size, and storage condition of the sample prior to the analysis. Some margin of error is present in the curve (or trace) reported above.

Any of the foregoing analytical techniques can be used alone or in combination to identify a particular form of compound of Formula (I). In addition, other methods of physical characterisation can also be employed to identify the characterised Form 2 compound of Formula (I). Examples of suitable techniques which are known to those skilled in the art to be useful for the physical characterisation of identification of a crystalline form or solvate include but are not limited to x-ray diffraction (XRD), infra-red spectroscopy (IR), Raman spectroscopy, differential scanning calorimetry (DSC) and solid state nuclear magnetic resonance spectroscopy (SSNMR). These techniques may be employed alone or in combination with other techniques to characterise a sample of an unknown form of the compound of Formula (I), and to distinguish Form 2 from other forms of compound of Formula (I).

The present invention includes Form 2 compound of Formula (I) both in substantially pure form and in admixture with other forms of compound of Formula (I). By “substantially pure” is meant that the composition comprises at least 90 percent Form 2 compound of Formula (I) as compared to the other forms of compound of Formula (I) in the composition, more particularly at least 95 percent Form 2 and in one aspect, at least 97 percent Form 2 compound of Formula (I).

### Pharmaceutical Compositions

Form 2 of a compound of Formula (I) will normally, but not necessarily, be formulated into pharmaceutical compositions prior to administration to a patient. In one aspect, the invention is directed to pharmaceutical compositions comprising Form 2 of a compound of Formula (I).

In another aspect, the invention is directed to a pharmaceutical composition comprising Form 2 of a compound of Formula (I) and one or more pharmaceutically acceptable excipients.

The excipient must be “acceptable” in the sense of being compatible with the other ingredients of the formulation and not deleterious to the recipient thereof.

The pharmaceutical compositions of the invention may be prepared and packaged in bulk form wherein a safe and effective amount of Form 2 of a compound of Formula (I) can be extracted and then given to the patient such as with tablets, capsules, powders in bottles or sachets or syrups (solutions or suspensions). Alternatively, the pharmaceutical compositions of the invention may be prepared and packaged in unit dosage form wherein each physically discrete unit contains a safe and effective amount of Form 2 of a compound of Formula (I). When prepared in unit dosage form, the pharmaceutical compositions of the invention typically contain from about 0.1 mg to 5000 mg, in another aspect from about 0.1 mg to 1000 mg, in a further aspect from about 0.1 mg to 100 mg, in a yet further aspect 0.1 mg to about 50 mg of Form 2 of a compound of Formula (I).

The pharmaceutical compositions of the invention typically contain Form 2 of a compound of Formula (I). However, in certain embodiments, the pharmaceutical compositions of the invention may optionally further comprise one or more additional active therapeutic compounds. The pharmaceutical compositions of the invention typically contain more than one pharmaceutically acceptable excipient. However, in certain embodiments, the pharmaceutical compositions of the invention contain one pharmaceutically acceptable excipient.

As used herein, the term “pharmaceutically acceptable” means suitable for pharmaceutical use.

Form 2 of a compound of Formula (I) and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carriage or transport of Form 2 of a compound of Formula (I) from one organ, or portion of the body, to another organ, or portion of the body, once administered to the patient. Certain pharmaceutically acceptable excipients may be chosen for their ability to enhance patient compliance.

Suitable pharmaceutically acceptable excipients include the following types of excipients: diluents, binders, disintegrants, lubricants, glidants, antiadherents, sorbents, granulating agents, coating agents, wetting agents, solvents, co-solvents, suspending agents, density modifiers, emulsifiers, sweeteners, flavouring agents, flavour masking agents, coloring agents, anticaking agents, humectants, chelating agents, plasticizers, viscosity increasing agents, reducing agents, antioxidants, preservatives, stabilizers, solubilizers, surfactants, isotonicity modifiers, bulking agents, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid or liquid oral dosage form such as a liquid, tablet, lozenge or a capsule, comprising a safe and effective amount of Form 2 of a compound of Formula (I) and an excipient. The excipient may be in the form of a diluent or filler. Suitable diluents and fillers in general include, but are not limited to lactose, sucrose, glucose, dextrose, mannitol, sorbitol, other polyols (or sugar alcohols), starch (e.g. corn starch, potato starch, and pre-gelatinized starch), cellulose and its derivatives (e.g. microcrystalline cellulose), calcium carbonate, calcium sulfate, and dibasic calcium phosphate. A liquid dosage form will generally consist of a suspension or solution of Form 2 of a compound of Formula (I) in a liquid excipient, either aqueous or non-aqueous, for example, ethanol, olive oil, glycerine, synthetic or natural mono or polyglyceride oils such as Myglyol (a commercially available medium chain triglyceride), glucose (syrup) or water (e.g. with an added flavouring, suspending, surface active or colouring agents). Where the composition is in the form of a tablet, capsule, caplet, pill, troche, powder, or lozenge, any pharmaceutical excipient routinely used for preparing solid formulations may be used. Examples of such excipients include lactose, sucrose, dextrose, mannitol, sorbitol, other polyols (or sugar alcohols), starch (e.g. corn starch, potato starch, and pre-gelatinized starch), cellulose and its derivatives (e.g. microcrystalline cellulose), calcium sulfate, and dibasic calcium phosphate magnesium stearate, terra alba, talc, gelatin, acacia, stearic acid, starch, cellulose, lactose and sucrose. Where the composition is in the form of a capsule, any routine encapsulation formulation is suitable, for example using the aforementioned excipients or a semi solid e.g. mono or di-glycerides of capric acid, Gelucire™ and Labrasol™, or a hard capsule shell e.g gelatin. Where the composition is in the form of a soft shell capsule e.g. gelatin, any pharmaceutical excipient routinely used for preparing dispersions or suspensions may be considered, for example aqueous gums or oils, and may be incorporated in a soft capsule shell.

An oral solid dosage form may further comprise an excipient in the form of a binder. Suitable binders include, but are not limited to, starch (e.g. corn starch, potato starch, and pre-gelatinized starch), sucrose, polyethylene glycol, gelatin, acacia, sodium alginate, alginic acid, tragacanth, guar gum, povidone, and cellulose and its derivatives (e.g. hydroxypropyl methyl cellulose, microcrystalline cellulose). The oral solid dosage form may further comprise an excipient in the form of a disintegrant. Suitable disintegrants include, but are not limited to, starch, cellulose, crospovidone, sodium starch glycolate, croscarmelose sodium, alginic acid, and sodium carboxymethyl cellulose. The oral solid dosage form may further comprise an excipient in the form of a lubricant. Suitable lubricants include, but are not limited to, stearic acid, magnesium stearate, calcium stearate, polyethylene glycol, sodium lauryl sulphate, sodium stearyl fumarate, talc and liquid paraffin.

There is further provided by the present invention a process of preparing a pharmaceutical composition, which process comprises mixing Form 2 of a compound of Formula (I), together with a pharmaceutically acceptable excipient.

### Composition A

The following Composition A was prepared by suspending ingredient (a) in aqueous solution containing ingredients (c), (d) and (b) in a bead milling machine to achieve sub-micron particulates. Ingredients (e), (f) and (g) were used as dispersant and processing aids. The final suspension was spray dried to yield a spray dried powder. The spray dried powder can be encapsulated by a gelatin capsule, the size of which is determined by the required dose size.

| | | |
|---|---|---|
| Component | Unit Formula (% w/w) | Function |

Suspension for bead milling

| | | |
|---|---|---|
| (a) Form 2 of compound of | 10.0 | Active Ingredient |
| Formula (I) | | |
| (b) Mannitol 60 | 9.0 | Vehicle |
| (c) Hypromellose 2910 | 1.0 | Vehicle |
| (d) Sodium Lauryl Sulfate | 0.2 | Wetting Agent |
| (e) Purified Water | 79.8* | Dispersant |
| (f) YTZ Grinding Beads | 3200.0 g** | Processing Aid |
| (g) Nitrogen | QS*** | Processing Aid |
| Total Suspension: | 100% | — |

Composition of Spray Dried Powder (Produced
from Spray Drying Suspension)

| | | |
|---|---|---|
| Form 2 of compound of | 49.5 | Active Ingredient |
| Formula (I) | | |
| Mannitol 60 | 44.5 | Vehicle |
| Hypromellose 2910 | 5.0 | Vehicle |
| Sodium Lauryl Sulfate | 1.0 | Wetting Agent |
| Spray Dried Powder | 100.0% | |
| Total: | | |

*Removed during spray drying process.
**A processing aid used as a grinding medium during wet bead milling of the suspension.
***Used during spray drying process.
Desiccant used: Sorb-It Silica Gel 2 Unit Bags (52 g).

Form 2 of a compound of Formula (I) may be administered by any suitable route of administration, including systemic administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes dermal application to the skin as well as intraocular, buccal (e.g. sub-lingually), rectal, intravaginal, and intranasal administration.

Preparations for oral administration may be suitably formulated to give controlled/extended release of Form 2 of a compound of Formula (I).

Form 2 of a compound of Formula (I) may be administered once only, or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. The dosage will also vary according to the nature of the intended treatment, for example a greater dose of compound may be given for amelioration as compared with prevention of a condition being treated. Suitable dosing regimens for Form 2 of a compound of Formula (I) depend on the pharmacokinetic properties of that compound, such as absorption, distribution, and half-life, which can be determined by the skilled artisan. In addition, suitable dosing regimens for Form 2 of a compound of Formula (I), including the duration such regimens are administered, depend on the route of administration of the compound, on the condition being treated, the severity of the condition being treated, the age and physical condition of the patient being treated, the medical history of the patient to be treated, the nature of any concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. It will also be appreciated that if Form 2 of a compound of Formula (I) is administered in combination with one or more additional active therapeutic agents as discussed further herein, the dosing regimen for Form 2 of a compound of Formula (I) may also vary according to the nature and amount of the one or more additional active therapeutic agents as necessary.

Typical daily dosages may vary depending upon the particular route of administration chosen. Typical daily dosages for oral administration range from about 0.01 to about 75 mg/kg, in one aspect from about 0.01 to about 25 mg/kg, in another aspect from about 0.1 to about 14 mg/kg. Typical daily dosages for parenteral administration range from about 0.001 to about 10 mg/kg; in one embodiment from about 0.01 to about 6 mg/kg. In one embodiment, the daily dose range of the compounds is from 100-1000 mg per day.

Form 2 of a compound of Formula (I) may also be used in combination with other active therapeutic agents. The invention thus provides, in a further aspect, a combination comprising Form 2 of a compound of Formula (I) together with a further active therapeutic agent. When Form 2 of the compound of Formula I is used in combination with a second active therapeutic agent which is active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The compounds of the present invention may be used alone or in combination with one or more additional active therapeutic agents, such as other antiparasitic drugs, for example antimalarial drugs.

Such other active therapeutic agents include antimalarial drugs such as chloroquine, mefloquine, primaquine quinine, artemisinin, halofantrine, doxycycline, amodiquine, atovaquone, tafenoquine, dapsone, proguanil, sulfadoxine, pyrimethamine, chlorcycloguanil, cycloguanil, and fansidar.

The combinations referred to above may conveniently be presented for use in the form of a pharmaceutical formulation and thus pharmaceutical formulations comprising a combination as defined above together with a pharmaceutically acceptable excipient comprise a further aspect of the invention. The individual components of such combinations may be administered either sequentially or simultaneously in separate or combined pharmaceutical formulations by any convenient route.

When administration is sequential, either the compound of the present invention or the one or more additional active therapeutic agent(s) may be administered first. When administration is simultaneous, the combination may be administered either in the same or different pharmaceutical composition. When combined in the same formulation it will be appreciated that the compound of the present invention and the one or more additional active therapeutic agent(s) must be stable and compatible with each other and the other components of the formulation. When formulated separately the compound of the present invention and the one or more additional active therapeutic agent(s) may be provided in any convenient formulation, conveniently in such manner as are known for such compounds in the art.

### EXAMPLES

The following Examples are intended for illustration only and are not intended to limit the scope of the invention in any way.

### Preparation of Form 2

Form 1 of the compound of Formula (I) may be prepared by the methods described in PCT Patent Application No. PCT/EP2007/055188, published as WO 2007/138048.

### Example 1

Form 1 of the compound of Formula (I) may be converted to Form 2 of the compound of Formula (I) using the following process of Scheme 1:

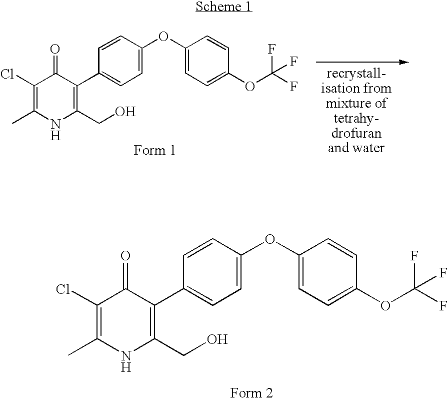

Form 2 can be prepared by re-crystallisation of Form 1 from a mixture of tetrahydrofuran and water. In particular this is a laboratory scale procedure using a crystallisation in tetrahydrofuran and water.

### Example 2

Form 1 (700 mg) was slurried in trifluoroethanol (10 ml) at ambient temperature for several hours, seeded with form 2 and aged for 24 h to give the desired polymorphic Form 2.

### Example 3

Form 1 (60 mg) was slurried in trifluoroethanol (1 ml) at 10 to 40° C. (temperature cycled) for 48 h to give the desired polymorphic Form 2.

### Example 4

Form 1 (200 g, 1.0 wt) was suspended in a mixture of tetrahydrofuran (884 ml, 4.42 vol) and water (180 ml, 0.90 vol). The suspension was heated to reflux to give a clear yellowish solution. The solution was inline-filtered into another reactor, resulting in some solid precipitation. The resulting suspension was heated to reflux resulting in dissolution of all solids. Above the solvent level solids started to be formed on the reactor walls. The mixture was maintained at 65° C. and tetrahydrofuran (52 ml, 0.26 vol) and water (11 ml, 0.055 vol) were added. The temperature was cooled slowly to 56° C. and seeds of Form 2 (1 g, 0.005 wt) were added as solids, and the resulting turbid solution was stirred for 0.5 h. At a temperature of 56° C., water (260 ml, 1.3 vol) was added over 165 min. The solution was kept for 30 min at 56° C. The suspension was cooled down to 0° C. via ramp over 3 h. The suspension was kept at 0° C. for another 10 hours. The mixture was filtered and the filter cake was washed with an acetone (380 ml, 1.9 vol)/tetrahydrofuran (40 ml, 0.2 vol) mixture and then acetone (2×420 ml, 2.1 vol). Drying at 50° C. in vacuum gave polymorphic Form 2 as a white solid (86% yield).

### Example 5

Form 1 (8.81 kg, 1.0 wt) was suspended in a mixture of tetrahydrofuran (41.5 L, 4.7 vol) and water (8.5 L, 0.96 vol). The suspension was heated to reflux to give a clear yellowish solution. The solution was inline-filtered into another reactor. The filtered solution was heated to 62° C. to achieve dissolution of all solids. The mixture was cooled to 60° C. and seeds of Form 2 (43 g, 0.005 wt) were added as a solid. The resulting suspension was stirred for 0.75 h at 58-59° C., and then water (11.4 L, 1.29 vol) was added over 117 min. The solution was kept for 87 min at 58° C. The suspension was cooled down to 0-5° C. via ramp over 4 h. The suspension was kept at 0-5° C. for another 9 hours. The mixture was filtered and the filter cake was washed with an acetone (17.0 L, 1.93 vol)/tetrahydrofuran (2.0 L, 0.22 vol) mixture and then acetone (2×19 L, 2.16 vol). Drying at 50° C. in vacuum gave polymorphic Form 2 as a white solid (83% yield).

### Raman Spectroscopy

Raman analysis was performed on a FT Raman spectrometer with a Microstage accessory. Approximately 5-20 mg of sample was placed on stainless steel or gold-plated sample cups. Slight pressure was applied to the top of the sample to pack the powder with a smooth surface.

Representative peaks observed in the Raman spectrum of Form 2 of compound of Formula (I) were as follows: 364, 414, 429, 587, 600, 642, 811, 1074, 1153, 1167, 1209, 1270, 1346, 1527, 1602, 1617, 2937, 3057, 3071 and 3087 cm⁻¹.

The margin of error in the foregoing peak assignments is approximately ±1 cm⁻¹. In one aspect, the margin of error in the foregoing peak assignments is ±1 cm⁻¹.

### X-Ray Powder Diffraction (XRD)

The diffraction pattern of FIG. 2 was obtained using copper Kα radiation on a Philips X'Pert Pro diffractometer equipped with a Philips X'Celerator Real Time Multi Strip (RTMS) detector. The sample was packed into a zero background holder and scanned from 2 to 40 °2θ using the following acquisition parameters: 40 mA, 45 kV, 0.02 °2θ step, 40 s step time. The sample was spun at 25 rpm during analysis.

A powder sample of Form 2 of compound of Formula (I) was used to produce the XRD pattern of FIG. 2.

Form 2 of compound of Formula (I) can be identified by certain characteristic 2 theta angle peaks at 5.0, 10.1, 14.2, 15.1, 16.4, 18.9, 19.6, 20.0, 25.4, 26.0, 26.5 and 28.0 degrees, which correspond respectively to d-spacings at 17.6, 8.8, 6.2, 5.8, 5.4, 4.7, 4.5, 4.4, 3.5, 3.4, 3.4 and 3.2 Angstroms (Å).

Further 2 theta angle peaks are at essentially the following positions: 17.7, 19.8, 20.3, 20.9, 22.5, 23.3, 23.6, 23.7, 33.9, 37.5, 39.1 and 40.3 degrees which correspond respectively to d-spacings at 5.0, 4.5, 4.4, 4.2, 3.9, 3.8, 3.8, 3.7, 2.6, 2.4, 2.3 and 2.2 Angstroms (Å).

The margin of error in the foregoing 2 theta angles is approximately ±0.1 degrees for each of the foregoing peak assignments. In one aspect, the margin of error in the foregoing 2 theta angles is ±0.1 degrees.

### Differential Scanning Calorimetry (DSC)

DSC was performed on a TA instruments Q1000 Differential Scanning Calorimeter equipped with a refrigerated cooling system. The sample was heated in a crimped aluminium pan from 25 to 300° C. using a heating rate of 15° C./min.

In respect of Form 2 of the compound of Formula (I), the margin of error is in the order of ±20° C. and ±10 J/g for the heat of fusion.

### Thermogravimetric Analysis (TGA)

TGA was performed TA Instruments Model Q500 system. The sample was placed in an open platinum pan.

The TGA of Form 2 of compound of Formula (I) displays negligible weight change between the ambient and 200° C., which is consistent with Form 2 being a non-solvated form.

1. A crystalline compound of Formula (I) 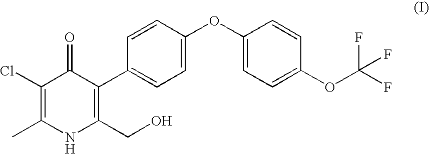
(Form 2), characterised by an XRD pattern expressed in terms of 2 theta angles and obtained with a diffractometer using copper Kα-radiation (45 kV/40 mA) at 0.02 °2θ step, according to the procedures described herein, wherein the XRD pattern comprises 2 theta angles (°2θ), with a margin of error of approximately ±0.1 degrees, at 5.0, 10.1, 14.2, 15.1, 16.4, 18.9, 19.6, 20.0, 25.4, 26.0, 26.5 and 28.0 degrees, which correspond respectively to d-spacings at 17.6, 8.8, 6.2, 5.4, 5.8, 4.7, 4.5, 4.4, 3.5, 3.4, 3.4 and 3.2 Angstroms (Å).

2. A crystalline compound of Formula (I) (Form 2) according to claim 1, further characterised in that it provides an XRD pattern expressed in terms of 2 theta angles and obtained with a diffractometer using copper Kα-radiation (45 kV/40 mA) at 0.02 °2 step, according to the procedures described herein, wherein the XRD pattern comprises 2 theta angles (°2θ), with a margin of error of approximately ±0.1 degrees, at 5.0, 10.1, 14.2, 15.1, 16.4, 17.7, 18.9, 19.6, 19.8, 20.0, 20.3, 20.9, 22.5, 23.3, 23.6, 23.7, 25.4, 26.0, 26.5, 28.0, 33.9, 37.5, 39.1, 40.3 degrees, which correspond respectively to d-spacings at 17.6, 8.8, 6.2, 5.8, 5.4, 5.0, 4.7, 4.5, 4.5, 4.4, 4.4, 4.2, 3.9, 3.8, 3.8, 3.7, 3.5, 3.4, 3.4, 3.2, 2.6, 2.4, 2.3 and 2.2 Angstroms (Å).

3. A crystalline compound of Formula (I) (Form 2) according to claim 1, further characterised in that it provides substantially the same X-ray powder diffraction (XRD) pattern as FIG. 2, wherein the XRD pattern is expressed in terms of 2 theta angles and obtained with a diffractometer using copper Kα-radiation (45 kV/40 mA) at 0.02 °2 step according to the procedures described herein.

4. A crystalline compound of Formula (I) 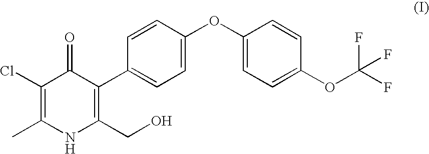
(Form 2), characterised by a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks, with a margin of error of approximately ±1 cm⁻¹, at 364, 414, 429, 587, 1074, 1270, 1527, 2937 and 3087 cm⁻¹.

5. A crystalline compound of Formula (I) (Form 2) according to claim 4, further characterised in that it provides a Raman spectrum obtained using an FT Raman spectrometer equipped with a 1064 nm excitation laser and a liquid nitrogen cooled Ge detector at spectral resolution of 4 cm⁻¹ according to the procedures described herein comprising peaks, with a margin of error of approximately ±1 cm⁻¹, at 364, 414, 429, 587, 600, 642, 811, 1074, 1153, 1167, 1209, 1270, 1346, 1527, 1602, 1617, 2937, 3057, 3071 and 3087 cm⁻¹.

6. A crystalline compound of Formula (I) (Form 2) according to claim 4, further characterised in that it provides substantially the same Raman spectrum as FIG. 1, wherein the Raman spectrum is obtained using a Fourier Transform (FT) Raman spectrometer equipped with a 1064 nm excitation laser and a liquid nitrogen cooled Ge detector at spectral resolution of 4 cm⁻¹ according to the procedures described herein.

7. A crystalline compound of Formula (I) (Form 2) characterised according to claims 1, and further characterised according to claim 4.

8. A crystalline compound of Formula (I) (Form 2) according to claim 1, further characterised in that it provides substantially the same thermogravimetric analysis (TGA) curve as FIG. 4, wherein the TGA was performed using open platinum pan at a heating rate of 15° C. per minute according to the procedures described herein.

9. A pharmaceutical composition comprising a crystalline compound of Formula (I) (Form 2) according to claim 1, and one or more pharmaceutically acceptable excipients.

10-12. (canceled)

13. A method for treating a subject suffering from a condition caused by infection by Plasmodium falciparum, comprising administering to the subject an effective amount of a crystalline compound of Formula (I) (Form 2) according to claim 1.

14. A method according to claim 13, wherein the subject is a human.
